SEQUENCE LISTING (64 sequences)

SEQ ID NO: 1 - DNA, 22 bp, Artificial Sequence
  Description of Artificial Sequence Synthetic primer
gaagtcccattgacacctttcc

SEQ ID NO: 2 - DNA, 25 bp, Artificial Sequence
  Description of Artificial Sequence Synthetic probe
cataaaggaatcctcatggccacaa

SEQ ID NO: 3 - DNA, 20 bp, Artificial Sequence
  Description of Artificial Sequence Synthetic primer
tgggtgagttggccaaatcc

SEQ ID NO: 4 - DNA, 21 bp, Artificial Sequence
  Description of Artificial Sequence Synthetic primer
ttgacacctttcccactgacc

SEQ ID NO: 5 - DNA, 24 bp, Artificial Sequence
  Description of Artificial Sequence Synthetic primer
gaagtcccattgacacctttccca

SEQ ID NO: 6 - DNA, 29 bp, Artificial Sequence
  Description of Artificial Sequence Synthetic probe
tgaccccataaaggaatcctcatggccac

SEQ ID NO: 7 - DNA, 18 bp, Artificial Sequence
  Description of Artificial Sequence Synthetic primer
ccgaagtcccattgacac

SEQ ID NO: 8 - DNA, 33 bp, Artificial Sequence
  Description of Artificial Sequence Synthetic probe
ctttcccactgaccccataaaggaatcctcatg

SEQ ID NO: 9 - DNA, 18 bp, Artificial Sequence
  Description of Artificial Sequence Synthetic primer
gagaaggagcccctggag

SEQ ID NO: 10 - DNA, 20 bp, Artificial Sequence
  Description of Artificial Sequence Synthetic probe
tagaccgagccgaagccgcc

SEQ ID NO: 11 - DNA, 18 bp, Artificial Sequence
  Description of Artificial Sequence Synthetic primer
ggcaagttgtcggagacg

SEQ ID NO: 12 - DNA, 25 bp, Artificial Sequence
  Description of Artificial Sequence Synthetic probe
cttcggctcggtctactcaggcatc

SEQ ID NO: 13 - DNA, 24 bp, Artificial Sequence
  Description of Artificial Sequence Synthetic probe
tgcctgagtagaccgagccgaagc

SEQ ID NO: 14 - DNA, 19 bp, Artificial Sequence
  Description of Artificial Sequence Synthetic probe
cggtctactcaggcatccg

SEQ ID NO: 15 - DNA, 20 bp, Artificial Sequence
  Description of Artificial Sequence Synthetic probe
cagtaccaggtgggcccgct

SEQ ID NO: 16 - DNA, 17 bp, Artificial Sequence
  Description of Artificial Sequence Synthetic probe
agcggcggcttcggctc

SEQ ID NO: 17 - DNA, 15 bp, Artificial Sequence
  Description of Artificial Sequence Synthetic probe
agccgaagccgccgc

SEQ ID NO: 18 - DNA, 19 bp, Artificial Sequence
  Description of Artificial Sequence Synthetic probe
cgggcccacctggtactgc

SEQ ID NO: 19 - DNA, 17 bp, Artificial Sequence
  Description of Artificial Sequence Synthetic primer
gcgcggcgaatctcaac

SEQ ID NO: 20 - DNA, 17 bp, Artificial Sequence
  Description of Artificial Sequence Synthetic primer
ggcccctgtagaggctt

SEQ ID NO: 21 - DNA, 27 bp, Artificial Sequence
  Description of Artificial Sequence Synthetic probe
caccagtttctctgctttccaccctgg

SEQ ID NO: 22 - DNA, 21 bp, Artificial Sequence
  Description of Artificial Sequence Synthetic probe
tctgctttccaccctggcgcc

SEQ ID NO: 23 - DNA, 17 bp, Artificial Sequence
  Description of Artificial Sequence Synthetic probe
ttccaccctggcgcccc

SEQ ID NO: 24 - DNA, 22 bp, Artificial Sequence
  Description of Artificial Sequence Synthetic probe
cactgaacccgctaagcccgca

SEQ ID NO: 25 - DNA, 24 bp, Artificial Sequence
  Description of Artificial Sequence Synthetic probe
ctgcgcagattgagcccactgaac

SEQ ID NO: 26 - DNA, 16 bp, Artificial Sequence
  Description of Artificial Sequence Synthetic probe
acccgctaagcccgca

SEQ ID NO: 27 - DNA, 26 bp, Artificial Sequence
  Description of Artificial Sequence Synthetic probe
agattgagcccactgaacccgctaag

SEQ ID NO: 28 - DNA, 16 bp, Artificial Sequence
  Description of Artificial Sequence Synthetic primer
ggacgctcggctcatg

SEQ ID NO: 29 - DNA, 18 bp, Artificial Sequence
  Description of Artificial Sequence Synthetic probe
cagcagccgccacgtccc

SEQ ID NO: 30 - DNA, 18 bp, Artificial Sequence
  Description of Artificial Sequence Synthetic primer
gaagcccatctcctcgca

SEQ ID NO: 31 - DNA, 14 bp, Artificial Sequence
  Description of Artificial Sequence Synthetic primer
ggacgctcggctca

SEQ ID NO: 32 - DNA, 23 bp, Artificial Sequence
  Description of Artificial Sequence Synthetic probe
cccttccgtcttgtcaaagacca

SEQ ID NO: 33 - DNA, 18 bp, Artificial Sequence
  Description of Artificial Sequence Synthetic probe
cggaagggacgtggcggc

SEQ ID NO: 34 - DNA, 22 bp, Artificial Sequence
  Description of Artificial Sequence Synthetic primer
ctcggctcatggtctttgacaa

SEQ ID NO: 35 - DNA, 21 bp, Artificial Sequence
  Description of Artificial Sequence Synthetic probe
agctgagtccggctaccctgg

SEQ ID NO: 36 - DNA, 18 bp, Artificial Sequence
  Description of Artificial Sequence Synthetic primer
ctgaggaagcccatctcc

SEQ ID NO: 37 - DNA, 27 bp, Artificial Sequence
  Description of Artificial Sequence Synthetic probe
ccacgtcccttccgtcttgtcaaagac

SEQ ID NO: 38 - DNA, 18 bp, Artificial Sequence
  Description of Artificial Sequence Synthetic probe
cgttggagcgcgaggagc

SEQ ID NO: 39 - DNA, 16 bp, Artificial Sequence
  Description of Artificial Sequence Synthetic primer
gacgctcggctcatgg

SEQ ID NO: 40 - DNA, 25 bp, Artificial Sequence
  Description of Artificial Sequence Synthetic probe
tttgacaagacggaagggacgtggc

SEQ ID NO: 41 - DNA, 21 bp, Artificial Sequence
  Description of Artificial Sequence Synthetic probe
tccggctaccctggcgttgga

SEQ ID NO: 42 - DNA, 18 bp, Artificial Sequence
  Description of Artificial Sequence Synthetic primer
gaaaccgactcggctgtg

SEQ ID NO: 43 - DNA, 15 bp, Artificial Sequence
  Description of Artificial Sequence Synthetic probe
cgccccagcgcacag

SEQ ID NO: 44 - DNA, 20 bp, Artificial Sequence
  Description of Artificial Sequence Synthetic primer
agccgaggagaaagaagcca

SEQ ID NO: 45 - DNA, 18 bp, Artificial Sequence
  Description of Artificial Sequence Synthetic probe
cagcaccagcgccccagc

SEQ ID NO: 46 - DNA, 17 bp, Artificial Sequence
  Description of Artificial Sequence Synthetic probe
tggtgctggcgggtggc

SEQ ID NO: 47 - DNA, 18 bp, Artificial Sequence
  Description of Artificial Sequence Synthetic primer
ggaagccgaggagaaaga

SEQ ID NO: 48 - DNA, 15 bp, Artificial Sequence
  Description of Artificial Sequence Synthetic probe
cgccccagcgcacag

SEQ ID NO: 49 - DNA, 15 bp, Artificial Sequence
  Description of Artificial Sequence Synthetic primer
cacccgccagcacca

SEQ ID NO: 50 - DNA, 17 bp, Artificial Sequence
  Description of Artificial Sequence Synthetic probe
ccacccgccagcaccag

SEQ ID NO: 51 - DNA, 20 bp, Artificial Sequence
  Description of Artificial Sequence Synthetic primer
ggaagccgaggagaaagaag

SEQ ID NO: 52 - DNA, 20 bp, Artificial Sequence
  Description of Artificial Sequence Synthetic primer
ttggacggctggtcctctat

SEQ ID NO: 53 - DNA, 33 bp, Artificial Sequence
  Description of Artificial Sequence Synthetic probe
catgttccccaggatggaagagacgatgaatga

SEQ ID NO: 54 - DNA, 23 bp, Artificial Sequence
  Description of Artificial Sequence Synthetic primer
tcccaagtccaaaagaagggatg

SEQ ID NO: 55 - DNA, 35 bp, Artificial Sequence
  Description of Artificial Sequence Synthetic probe
caaagtaatgaagaggtggttcaactttctccaga

SEQ ID NO: 56 - DNA, 35 bp, Artificial Sequence
  Description of Artificial Sequence Synthetic probe
ctgaaagctcaaagtaatgaagaggtggttcaact

SEQ ID NO: 57 - DNA, 35 bp, Artificial Sequence
  Description of Artificial Sequence Synthetic probe
aggtggttcaactttctccagatgaggaaactaag

SEQ ID NO: 58 - DNA, 35 bp, Artificial Sequence
  Description of Artificial Sequence Synthetic probe
tctccagatgaggaaactaaggacctcattcatcg

SEQ ID NO: 59 - DNA, 29 bp, Artificial Sequence
  Description of Artificial Sequence Synthetic probe
aggtcactcagacagtccctcacatgttc

SEQ ID NO: 60 - DNA, 28 bp, Artificial Sequence
  Description of Artificial Sequence Synthetic probe
ctcagacagtccctcacatgttccccag

SEQ ID NO: 61 - DNA, 26 bp, Artificial Sequence
  Description of Artificial Sequence Synthetic probe
atgaagaggtggttcaactttctcca

SEQ ID NO: 62 - DNA, 10 bp, Artificial Sequence
  Description of Artificial Sequence Synthetic probe
aaacacgtgc

SEQ ID NO: 63 - DNA, 10 bp, Artificial Sequence
  Description of Artificial Sequence Synthetic probe
ccttgttcca

SEQ ID NO: 64 - DNA, 10 bp, Artificial Sequence
  Description of Artificial Sequence Synthetic probe
cagggacgat

(19) United States
(12) Patent Application Publication
(10) Pub. No.: US 2011/0256537 A1
(43) Pub. Date: Oct. 20, 2011

(54) OPTIMIZED OLIGONUCLEOTIDES AND METHODS OF USING SAME FOR THE SCREENING, DETECTION, ISOLATION, QUANTITATION, MONITORING AND SEQUENCING OF PROSTATE CANCER ASSOCIATED VIRUSES AND HOST BIOMARKERS

(75) Inventors: Alice A. Jacobs, Cambridge, MA (US); Chesley Leslin, Boston, MA (US); David L. Dolinger, Medway, MA (US)

(73) Assignee: Intelligent Medical Devices, Inc.

(21) Appl. No.: 13/085,303

(22) Filed: Apr. 12, 2011

Related U.S. Application Data
(60) Provisional application No. 61/323,628, filed on Apr. 13, 2010.

(51) Int. Cl. C12Q 1/68; C12N 9/22; C07H 1/00; C07H 1/06; C12P 19/34; C07H 21/04

(52) U.S. Cl. 435/6.11; 435/6.12; 435/91.2; 536/23.1; 536/25.3; 536/24.33; 435/199; 536/24.3

(57) ABSTRACT

Described herein are oligonucleotides useful for screening, detecting, isolating, quantitating, monitoring and sequencing of viruses and host biomarkers associated with prostate cancer and methods and kits of using the described oligonucleotides.

### RELATED APPLICATION

This application claims the benefit of U.S. Provisional Application No. 61/323,628, filed on Apr. 13, 2010, the contents of which are incorporated by reference herein in their entirety.

### FIELD OF THE INVENTION

The present invention relates to oligonucleotides for screening, detecting, isolating, quantitating, monitoring and sequencing of viruses and host biomarkers associated with prostate cancer and methods for using the oligonucleotides.

### BACKGROUND

Prostate cancer is the most common form of non-skin cancer affecting U.S. males and carries a lifetime risk of developing in 1 in 6 adult U.S. males. Prostate cancer accounts annually for 3% of male deaths worldwide. The disease affects mainly older men, so, as men live longer, their risk for developing prostate cancer increases. Prostate cancer etiology may include infectious agents, such as viruses. Mounting scientific evidence suggests that viruses, such as BK virus, may be a contributing factor for at least some forms of prostate cancer.

A sensitive, accurate assay for detecting and screening for prostate cancer associated viruses and host biomarkers, would significantly aid in proper diagnosis and treatment of patients afflicted with prostate cancer.

BK Virus is a member of the polyomavirus family that infects approximately 80% of adults in the United States. The virus usually persists in an asymptomatic state. However, in immunocompromised individuals, BK can reactivate and cause serious disease. Studies have suggested that BK is associated with early stages of prostate cancer.

EBV is a member of the human herpes virus family and one of the most prevalent viruses currently known. EBV may have an infection rate of 95% worldwide and can persist latently in the body until activated. EBV can cause lymphoproliferative disorders in adults and infectious mononucleosis in adolescents and young adults. Activated EBV may also function to decrease the host cell-mediated immune response, an important defense against viral infection. Activated EBV has also been postulated to play a role in several cancers, including Burkitt's lymphoma. Consequently, increased EBV activity may indicate an increased risk for cancer.

Ribonuclease L (RNAse L) is a component of the human innate antiviral response. RNAse L degrades viral single-stranded RNA. The activated form of RNAse L also triggers a mitochondrial pathway of apoptosis, whereby virus-infected cells are eliminated. Individuals with mutations in the gene encoding RNAse L are more susceptible to viral infection.

One host biomarker, Prostatic Acid Phosphatase (PAP), that indicates the presence of prostate cancer may enhance infection by viruses. Prostatic Acid Phosphatase (PAP) is a nonspecific protein phosphatase produced in the prostate and secreted in semen. Increased levels of PAP have been found in prostate cancer patients. Fragments of PAP form amyloid fibrils that enhance viral infection by promoting viral attachment and entry.

Other host biomarkers are commonly used to indicate the presence of prostate cancer. Pim-1 and Pim-2 are serine/threonine kinases that may play a role in development of prostate cancer. Pim-1 and Pim-2 regulate genes involved in protein synthesis. Overexpression of Pim-1 and Pim-2 occurs in malignant cells. Increased protein synthesis mediated by Pim-1 and Pim-2 correlates with the observed rapid growth of malignant cells. Hepsin is a membrane-bound transmembrane serine protease. This protein is expressed in cells throughout the body and may play an essential role in maintaining cell morphology. Hepsin is overexpressed in malignant cells and has been used as a biomarker for prostate cancer. Prostate-specific membrane antigen (PSMA) is a membrane glycoprotein that is preferentially expressed on the surface of malignant cells. PSMA has folate hydrolase activity, but its functional cellular role is unknown

There exists a need for biomarkers to effectively diagnose prostate cancer. Also, if prostrate cancer is either caused by or enhanced by an infectious agent, such as BK Virus and/or EBV, it would be beneficial to detect that specific infectious agent. There is a particular need for a comprehensive, rapid and accurate molecular diagnostic test for screening of individuals at risk of developing prostate cancer. Such a test should involve the detection of both specific host markers (e.g. PAP) and infectious agents such as BK Virus and/or EBV that are associated with prostate cancer.

A rapid and accurate diagnostic test for the screening and/or detection of prostate cancer biomarkers, along with viral infectious agents, therefore, would provide clinicians with an effective tool for identifying patients at risk for developing cancer, thereby, subsequently supporting effective treatment regimens.

### SUMMARY

The present invention relates to oligonucleotides for screening, detecting, isolating, quantifying, monitoring and sequencing of RNAseL, PAP, Pim-1, Pim-2, Hepsin, PSMA, (prostate cancer associated biomarkers) and BK Virus and EBV (prostate cancer associated viruses). In one embodiment, the present invention is directed to an isolated polynucleotide, comprising a nucleotide sequence selected from the group consisting of SEQ ID NOS: 1-61.

In another embodiment, the present invention is directed to a method of hybridizing one or more isolated nucleic acid sequences comprising a sequence selected from the group consisting of SEQ ID NOS: 1-61 to RNAse L, PAP, Pim-1, Pim-2, Hepsin, PSMA, BK Virus and EBV, comprising contacting one or more isolated nucleic acid sequences to a sample comprising the RNAse L, PAP, Pim-1, Pim-2, Hepsin, PSMA, BK Virus and EBV sequence under conditions suitable for hybridization. In a particular embodiment, the RNAse L, PAP, Pim-1, Pim-2, Hepsin, PSMA, BK Virus and EBV sequence is a genomic sequence, a template sequence or a sequence derived from an artificial construct. In a particular embodiment, the method(s) further comprises screening, detecting, isolating, quantitating, monitoring and/or sequencing of the hybridized RNAse L, PAP, Pim-1, Pim-2, Hepsin, PSMA, BK Virus and EBV sequence.

Another embodiment is directed to a primer set comprising at least one forward primer selected from the group consisting of SEQ ID NOS: 1, 4, 5, 7, 9, 19, 28, 31, 34, 39, 42 and 52 and at least one reverse primer selected from the group consisting of SEQ ID NOS: 3, 11, 20, 30, 36, 44, 47, 49, 51 and 54. In a particular embodiment, the primer set is selected from the group consisting of: Groups 1-40 of Table 2.

In a particular embodiment, the present invention is directed to a primer comprising a sequence selected from the group consisting of SEQ ID NOS: 1, 3, 4, 5, 7, 9, 11, 19, 20, 28, 30, 31, 34, 36, 39, 42, 44, 47, 49, 51, 52 and 54 for the production of cDNA from RNAse L, PAP, Pim-1, Pim-2, Hepsin, PSMA, BK Virus and EBV RNA.

Another embodiment is directed to a method of producing a nucleic acid product, comprising contacting one or more isolated nucleic acid sequences selected from the group consisting of SEQ ID NOS: 1, 3, 4, 5, 7, 9, 11, 19, 20, 28, 30, 31, 34, 36, 39, 42, 44, 47, 49, 51, 52 and 54 to a sample selected from the group consisting of: a RNAseL, PAP, Pim-1, Pim-2, Hepsin, PSMA, BK Virus and EBV sequence under conditions suitable for nucleic acid polymerization. In a particular embodiment, the nucleic acid product is an amplicon produced using at least one forward primer selected from the group consisting of SEQ ID NOS: 1, 4, 5, 7, 9, 19, 28, 31, 34, 39, 42 and 52 and at least one reverse primer selected from the group consisting of SEQ ID NOS: 3, 11, 20, 30, 36, 44, 47, 49, 51 and 54.

Another embodiment is directed to a probe that hybridizes to an amplicon produced as described herein, e.g., using the primers described herein. In a particular embodiment, the probe comprises a sequence selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61. In a particular embodiment, the probe is labeled with a detectable label selected from the group consisting of: a fluorescent label, a chemiluminescent label, a quencher, a radioactive label, biotin, mass tag and/or gold. The probe may also be labeled with other similar detectable labels used in conjunction with probe technology as known by one of ordinary skill in the art.

Another embodiment is directed to a method for detecting RNAse L, PAP, Pim-1, Pim-2, Hepsin, PSMA, BK Virus or EBV DNA in a sample, comprising: a) contacting the sample with at least one forward primer comprising a sequence selected from the group consisting of SEQ ID NOS: 1, 4, 5, 7, 9, 19, 28, 31, 34, 39, 42 and 52, and at least one reverse primer comprising a sequence selected from the group consisting of SEQ ID NOS: 3, 11, 20, 30, 36, 44, 47, 49, 51 and 54 under conditions such that nucleic acid amplification occurs to yield an amplicon; and b) contacting the amplicon with one or more probes comprising one or more sequences selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61 under conditions such that hybridization of the probe to the amplicon occurs, wherein hybridization of the probe is indicative of RNAse L, PAP, Pim-1, Pim-2, Hepsin, PSMA, BK Virus or EBV in the sample. In a particular embodiment, each of the one or more probes is labeled with a different detectable label. In a particular embodiment, the one or more probes are labeled with the same detectable label. In a particular embodiment, the sample is selected from the group consisting of: blood, urine, serum, plasma, neoplastic or other tissue obtained from biopsies and samples specifically obtained from the prostate, cerebrospinal fluid, and the central nervous system. In a particular embodiment, the sample is from a human, is non-human in origin, or is derived from an inanimate object. In a particular embodiment, the at least one forward primer, the at least one reverse primer and the one or more probes are selected from the group consisting of: Groups 1-40 of Table 2. In a particular embodiment, the method(s) further comprise quantitating and/or sequencing RNAse L, PAP, Pim-1, Pim-2, Hepsin, PSMA, BK Virus or EBV RNA or DNA in a sample.

In another embodiment, the present invention is directed to a method for detecting, quantitating and grouping RNAse L, PAP, Pim-1, Pim-2, Hepsin , PSMA, BK Virus or EBV RNA in a sample, comprising: a) contacting the RNAse L, PAP, Pim-1, Pim-2, Hepsin, PSMA, BK Virus or EBV RNA with a reverse transcriptase primer sequence selected from the group consisting of SEQ ID NOS: 1, 3, 4, 5, 7, 9, 11, 19, 20, 28, 30, 31, 34, 36, 39, 42, 44, 47, 49, 51, 52 and 54 for the production of cDNA; b) contacting the cDNA with at least one forward primer comprising the sequence selected from the group consisting of SEQ ID NOS: 1, 4, 5, 7, 9, 19, 28, 31, 34, 39, 42 and 52 and at least one reverse primer sequence selected from the group consisting of SEQ ID NOS: 3, 11, 20, 30, 36, 44, 47, 49, 51 and 54 under conditions such that nucleic acid amplification occurs to yield an amplified PCR product; and c) contacting the amplified PCR product with one or more probes comprising one or more sequences selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61 under conditions such that binding occurs; wherein detection, quantification and grouping of an amplified PCR product by one or more probes is indicative of the presence of one or more of the groups of RNAseL, PAP, Pim-1, Pim-2, Hepsin, PSMA, BK Virus or EBV in the sample. Step c) is performed using a labeled probe(s) comprising a sequence that hybridizes to the amplicon generated by the forward and reverse primer pair group of step b).

Another embodiment is directed to a primer set or collection of primer sets (at least one forward and reverse primer pair) for amplifying DNA from RNAseL, PAP, Pim-1, Pim-2, Hepsin, PSMA, BK Virus or EBV comprising a nucleotide sequence selected from the group consisting of: (1) SEQ ID NOS: 1 and 3; (2) SEQ ID NOS: 4 and 3; (3) SEQ ID NOS: 5 and 3; (4) SEQ ID NOS: 7 and 3; (5) SEQ ID NOS: 9 and 11; (6) SEQ ID NOS: 19 and 20; (7) SEQ ID NOS: 28 and 30; (8) SEQ ID NOS: 31 and 30; (9) SEQ ID NOS: 34 and 36; (9) SEQ ID NOS: 39 and 30; (10) SEQ ID NOS: 34 and 30; (11) SEQ ID NOS: 42 and 44; (12) SEQ ID NOS: 42 and 47; (13) SEQ ID NOS: 42 and 49; (14) SEQ ID NOS: 42 and 51; and (15) SEQ ID NOS: 52 and 54. A particular embodiment is directed to oligonucleotide probes for binding to RNAse L, PAP, Pim-1, Pim-2, Hepsin, PSMA, BK Virus or EBV RNA or DNA comprising a nucleotide sequence selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61.

Another embodiment is directed to a kit for detecting and quantitating RNAseL, PAP, Pim-1, Pim-2, Hepsin, PSMA, BK Virus or EBV RNA or DNA in a sample, comprising one or more probes comprising a sequence selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61.

In a particular embodiment, the kit further comprises: a) at least one forward primer comprising the sequence selected from the group consisting of SEQ ID NOS: 1, 4, 5, 7, 9, 19, 28, 31, 34, 39, 42 and 52; and b) at least one reverse primer comprising the sequence selected from the group consisting of SEQ ID NOS: 3, 11, 20, 30, 36, 44, 47, 49, 51 and 54. In a particular embodiment, the kit further comprises reagents for quantitating and/or sequencing RNAse L, PAP, Pim-1, Pim-2, Hepsin, PSMA, BK Virus or EBV DNA in the sample. In a particular embodiment, the one or more probes are labeled with different detectable labels. In a particular embodiment, the one or more probes are labeled with the same detectable label. In a particular embodiment, the at least one forward primer and the at least one reverse primer are selected from the group consisting of: Groups 1-40 of Table 2.

Another embodiment is directed to a kit for detecting, quantitating and sequencing RNAse L, PAP, Pim-1, Pim-2, Hepsin, PSMA, BK Virus or EBV RNA in a sample, comprising: a) at least one reverse transcriptase primer comprising a sequence selected from the group consisting of SEQ ID NOS: 1, 3, 4, 5, 7, 9, 11, 19, 20, 28, 30, 31, 34, 36, 39, 42, 44, 47, 49, 51, 52 and 54 for cDNA production and nucleic acid amplification; b) at least one forward primer comprising the sequence selected from the group consisting of SEQ ID NOS: 1, 4, 5, 7, 9, 19, 28, 31, 34, 39, 42 and 52 for nucleic acid amplification and at least one reverse primer sequence selected from the group consisting of SEQ ID NOS: 3, 11, 20, 30, 36, 44, 47, 49, 51 and 54 and c) one or more probes comprising a sequence selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61 for binding to an amplified nucleic acid product.

Another embodiment is directed to a kit for detecting, quantitating and sequencing a targeted RNAse L, PAP, Pim-1, Pim-2, Hepsin, PSMA, BK Virus or EBV sequence derived from an artificial construct, such as a plasmid, comprising: a) at least one forward primer comprising the sequence selected from the group consisting of SEQ ID NOS: 1, 4, 5, 7, 9, 19, 28, 31, 34, 39, 42 and 52; b) at least one reverse primer comprising the sequence selected from the group consisting of SEQ ID NOS: 3, 11, 20, 30, 36, 44, 47, 49, 51 and 54; and c) one or more probes comprising a sequence selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61.

Another embodiment is directed to a method of diagnosing prostate cancer, comprising: a) contacting a sample with at least one forward and reverse primer set selected from the group consisting of: Groups 1-40 of Table 2; b) conducting an amplification reaction, thereby producing an amplicon; and c) detecting the amplicon using one or more probes selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61; wherein the detection of an amplicon is indicative of the presence of RNAseL, PAP, Pim-1, Pim-2, Hepsin, PSMA, BK Virus or EBV in the sample. A further embodiment comprises reagents for sequencing RNAseL, PAP, Pim-1, Pim-2, Hepsin, PSMA, BK Virus or EBV DNA. In a particular embodiment, the sample is selected from the group consisting of: blood, urine, serum, plasma, neoplastic or other tissue obtained from biopsies and samples specifically obtained from the prostate, cerebrospinal fluid, and the central nervous system. In a particular embodiment, the sample is from a human, is non-human in origin, or is derived from an inanimate object.

### DETAILED DESCRIPTION

Viral infection may play a role in the development of prostate cancer. BK Virus has infected approximately 80% of the human population and usually exists in an asymptomatic state, but it can be reactivated in immunocompromised or immunosuppressed individuals. The virus can infect the urinary tract and produces tumor antigens that inactivate host tumor suppressors. A recent study suggests that BK Virus is associated with early stage prostate carcinomas. (Das et al., J. Virol, 82(6): 2705-2714, 2008). BK Virus activation is determined empirically by increased RNA expression.

RNAseL is a component of the innate antiviral immune response. An increased cancer risk may be associated with individuals who have an amino acid substitution (R462Q) in the RNAse L protein due to a single nucleotide polymorphism. RNAse L is an enzyme that degrades viral single-stranded RNA. By diminishing the activity of RNAse L, the R462Q mutation consequently reduces an individual's innate antiviral immune response. (Urisman et al., PLoS Pathogens, 2(3):0211, 2006). Other mutations in the RNAse L gene also contribute to decreased antiviral enzymatic activity that may subsequently play a role in prostate cancer etiology. (See, e.g., Madsen et al., PLoS One, 3(6):e2492, 2008).

An opportunistic viral pathogen, such as the latent Epstein-Barr virus (EBV) may be activated and infect cells since the innate immune response is compromised in an individual harboring a mutated RNAseL gene. EBV is a ubiquitous virus that has been estimated to infect over 95% of the world's population. EBV is benign for most people and remains in a latent state. However, upon activation, EBV can cause mononucleosis, lymphoproliferative disorders and may play a role in prostate cancer. EBV activation is determined empirically by increased RNA expression.

Host biomarkers, including prostate specific membrane antigen (PSMA), have been used to diagnose prostate cancer. PAP, PSMA, Pim-1, Pim-2 and Hepsin expression levels are substantially increased in malignant prostatic cells. Increased RNA levels for each of these genes can indicate prostate cancer. (See Landers et al., Int. J. Cancer, 114, 950-56, 2005; Chen et al., Mol. Cancer Res., 3(8):443-451, 2005; Hong et al., J. Virol., 83(14):6995-7003, 2009). One embodiment of the present invention, inter alia, is directed toward quantifying RNA levels of these genes to determine if there are increased expression levels in tested cells, blood, serum, plasma, feces, biopsy material, semen and urine.

The oligonucleotides of the present invention can be used in a multiplex format for the detection and quantitation of PAP and/or Hepsin and/or Pim-1 and/or Pim-2 and/or PSMA and/or BK Virus and/or EBV RNA as well as detection of a RNAseL mutation. Although some tests exist that can detect and/or quantitate each of these nucleic acids, a method for simultaneously identifying and quantifying (BK Virus and/or EBV) and (PAP and/or PSMA and/or Pim-1 and/or Pim-2 and/or Hepsin) genetic material and an RNAse L gene mutation is not presently available.

The present invention can be used to evaluate primers/probes against new sequence entries to ensure that they are still effective or indicate when a redesign is needed.

The present invention includes primer and probe sequences which are uniquely designed to generate a specific amplicon if the target nucleic acid is present. The probe specifically binds to this amplicon and is cleaved to generate a detectable fluorescent signal. The internal control multiplex is made up of complex multiplex sets (that include the internal control) consisting of multiple singleplex sets that do not heterodimerize with each other and can work together without negatively impacting each of the assays. All of the oligonucleotides that make up the sets are equally important to the invention.

Described herein are optimized oligonucleotides that can act as probes and primers that, alone or in various combinations, allow for the detection, isolation, quantitation, monitoring and sequencing of RNAseL, PAP, Pim-1, Pim-2, Hepsin, PSMA, BK Virus or EBV. Nucleic acid primers and probes for detecting genetic material of RNAseL, PAP, Pim-1, Pim-2, Hepsin, PSMA, BK Virus or EBV and methods for designing and optimizing the respective primer and probe sequences are described.

The primers and probes described herein can be used, for example, to confirm suspected cases of prostate cancer, symptoms, disorders or conditions in a singleplex format by determining if expression levels of the prostate cancer biomarkers are above or below a predetermined threshold level.

The primers and probes of the present invention can be used for the detection or screening of only RNAseL or PAP or Pim-1 or Pim-2 or Hepsin or PSMA or BK Virus or EBV or combined in a multiplex format without loss of assay precision or sensitivity. The multiplex format option allows relative comparisons to be made between various prevalent pathogens and host prostate cancer biomarkers. The primers and probes described herein can be used as a diagnostic reagent for prostate cancer. Moreover, the multiplex assay can be used to determine expression levels of multiple host biomarkers to provide a more accurate assessment of prostate cancer risk/detection than detecting the expression level of a single biomarker.

The primers and probe(s) (e.g., used to detect PAP) described herein have the unique feature of providing a lower rate of false positive and false negative results when used in diagnostic assays.

The advantages of a random access multiplex test format or multiplex format with the present invention are: (1) simplified and improved testing and analysis; (2) increased efficiency and cost-effectiveness; (3) the relationship between viruses (such as BK and EBV) and the prostate cancer biomarkers can provide more information regarding the clinical state of the patient; (4) decreased turnaround time (increased speed of reporting results); (5) increased productivity (less equipment time needed); (6) coordination/standardization of results for patients for multiple organisms (reduces error from inter-assay variation) and (7) prognostic value—the viral burden of each virus can be assessed. Moreover, a multiplex test indicates the expression levels of the prostate cancer biomarkers and/or viral RNA, enabling a better indication of the health of the patient and possibly the patient's stage of prostate cancer upon diagnosis, thereby rendering treatment earlier.

The present invention provides one or more sets of primers that can anneal to RNAseL, PAP, Pim-1, Pim-2, Hepsin, PSMA, BK Virus or EBV and thereby amplify a target from a biological sample. The present invention provides, for example, at least a first primer and at least a second primer for RNAseL, PAP, Pim-1, Pim-2, Hepsin, PSMA, BK Virus or EBV, each of which comprises a nucleotide sequence designed according to the inventive principles disclosed herein, which are used together to amplify RNAseL, PAP, Pim-1, Pim-2, Hepsin, PSMA, BK Virus or EBV DNA in a sample in a singleplex assay, or in a sample in a multiplex assay.

Also provided herein are probes that hybridize to the RNAseL, PAP, Pim-1, Pim-2, Hepsin, PSMA, BK Virus or EBV sequences and/or amplified products derived from the RNAseL, PAP, Pim-1, Pim-2, Hepsin, PSMA, BK Virus or EBV genes. A probe can be labeled, for example, such that when it binds to an amplified or unamplified target sequence, or after it has been cleaved after binding, a fluorescent signal is emitted that is detectable under various spectroscopy and light measuring apparatuses. The use of a labeled probe, therefore, can enhance the sensitivity of detection of a target in an amplification reaction of RNAseL, PAP, Pim-1, Pim-2, Hepsin, PSMA, BK Virus or EBV DNA because it permits the detection of host-derived DNA at low template concentrations that might not be conducive to visual detection as a gel-stained amplification product.

Primers and probes are sequences that anneal to a prostate cancer biomarker or viral genomic or prostate cancer biomarker or viral genomic derived sequence, e.g., PAP sequence, (the “target” sequence). The target sequence can be, for example, a prostate cancer biomarker or viral gene or a subset, “region”, of, in this case, a prostate cancer biomarker or viral gene. In one embodiment, the entire genomic sequence can be “scanned” for optimized primers and probes useful for detecting and/or quantitating the prostate cancer biomarker or viral gene. In other embodiments, particular regions of the prostate cancer biomarker or viral genome can be scanned, e.g., regions that are documented in the literature as being useful for detecting multiple prostate cancer biomarker alleles or viral gene, regions that are conserved, or regions where sufficient information is available in, for example, a public database, with respect to prostate cancer biomarker alleles or viral strains.

Sets or groups of primers and probes are generated based on the target to be detected. The set of all possible primers and probes can include, for example, sequences that include the variability at every site based on the known prostate cancer biomarker or viral gene, or the primers and probes can be generated based on a consensus sequence of the target. The primers and probes are generated such that the primers and probes are able to anneal to a particular strain or sequence under high stringency conditions. For example, one of ordinary skill in the art recognizes that for any particular sequence, it is possible to provide more than one oligonucleotide sequence that will anneal to the particular target sequence, even under high stringency conditions. The set of primers and probes to be sampled includes, for example, all such oligonucleotides for all prostate cancer biomarkers or viral gene sequences. Alternatively, the primers and probes include all such oligonucleotides for a given consensus sequence for a target.

Typically, stringent hybridization and washing conditions are used for nucleic acid molecules over about 500 bp. Stringent hybridization conditions include a solution comprising about 1 M Na⁻ at 25° C. to 30° C. below the Tm; e.g., 5×SSPE, 0.5% SDS, at 65° C.; (see, Ausubel, et al., Current Protocols in Molecular Biology, Greene Publishing, 1995; Sambrook et al., Molecular Cloning: A Laboratory Manual, Cold Spring Harbor Press, 1989). Tm is dependent on both the G+C content and the concentration of salt ions, e.g., Na⁺ and K⁻. A formula to calculate the Tm of nucleic acid molecules greater than about 500 by is Tm=81.5+0.41(%(G+C))−log₁₀[Na⁺]. Washing conditions are generally performed at least at equivalent stringency conditions as the hybridization. If the background levels are high, washing can be performed at higher stringency, such as around 15° C. below the Tm.

The set of primers and probes, once determined as described above, are optimized for hybridizing to a plurality of prostate cancer biomarkers or viral genes by employing scoring and/or ranking steps that provide a positive or negative preference or “weight” to certain nucleotides in a target nucleic acid strain sequence. If a consensus sequence is used to generate the full set of primers and probes, for example, then a particular primer sequence is scored for its ability to anneal to the corresponding sequence of every known native prostate cancer biomarker or viral gene sequence. Even if a probe were originally generated based on a consensus, therefore, the validation of the probe is in its ability to specifically anneal and detect every, or a large majority of, prostate cancer biomarker or viral gene sequences. The particular scoring or ranking steps performed depend upon the intended use for the primer and/or probe, the particular target nucleic acid sequence, and the number of strains of that target nucleic acid sequence.

The methods of the invention provide optimal primer and probe sequences because they hybridize to all or a subset of alleles of the prostate cancer biomarkers. Once optimized oligonucleotides are identified that can anneal to prostate cancer biomarkers, the sequences can then further be optimized for use, for example, in conjunction with another optimized sequence as a “primer set” or for use as a probe. A “primer set” is defined as at least one forward primer and one reverse primer.

Described herein are methods for using RNAseL, PAP, Pim-1, Pim-2, Hepsin, PSMA, BK Virus or EBV primers and probes for producing a nucleic acid product, for example, comprising contacting one or more nucleic acid sequences of SEQ ID NOS: 1-61 to a sample comprising at least one of the alleles of RNAseL, PAP, Pim-1, Pim-2, Hepsin, PSMA, BK Virus or EBV under conditions suitable for nucleic acid polymerization. The primers and probes can additionally be used to quantitate and/or sequence DNA or RNA of RNAseL, PAP, Pim-1, Pim-2, Hepsin, PSMA, BK Virus or EBV, or used as diagnostics to, for example, detect RNAseL, PAP, Pim-1, Pim-2, Hepsin, PSMA, BK Virus or EBV in a sample, e.g., obtained from a subject, e.g., a mammalian subject. Particular combinations for amplifying RNAseL, PAP, Pim-1, Pim-2, Hepsin, PSMA, BK Virus or EBV DNA include, for example, using at least one forward primer selected from the group consisting of SEQ ID NOS: 1, 4, 5, 7, 9, 19, 28, 31, 34, 39, 42 and 52 and at least one reverse primer selected from the group consisting of SEQ ID NOS: 3, 11, 20, 30, 36, 44, 47, 49, 51 and 54.

Methods are described for detecting RNAseL, PAP, Pim-1, Pim-2, Hepsin, PSMA, BK Virus or EBV in a sample, for example, comprising (1) contacting at least one forward and reverse primer set, e.g., SEQ ID NOS: 1, 4, 5, 7, 9, 19, 28, 31, 34, 39, 42 and 52 (forward primers) and SEQ ID NOS: 3, 11, 20, 30, 36, 44, 47, 49, 51 and 54 (reverse primers) to a sample; (2) conducting an amplification; and (3) detecting the generation of an amplified product, wherein the generation of an amplified product indicates the presence of RNAseL, PAP, Pim-1, Pim-2, Hepsin, PSMA, BK Virus or EBV in the sample.

The detection of amplicons using probes described herein can be performed, for example, using a labeled probe, e.g., the probe comprising a nucleotide sequence selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61, that hybridizes to one of the strands of the amplicon generated by at least one forward and reverse primer set. The probe(s) can be, for example, fluorescently labeled, thereby indicating that the detection of the probe involves measuring the fluorescence of the sample of the bound probe, e.g., after bound probes have been isolated. Probes can also be fluorescently labeled in such a way, for example, such that they only fluoresce upon hybridizing to their target, thereby eliminating the need to isolate hybridized probes. The probe can also comprise a fluorescent reporter moiety and a quencher of fluorescence moiety. Upon probe hybridization with the amplified product, the exonuclease activity of a DNA polymerase can be used to cleave the probe reporter and quencher, resulting in the unquenched emission of fluorescence, which is detected. An increase in the amplified product causes a proportional increase in fluorescence, due to cleavage of the probe and release of the reporter moiety of the probe. The amplified product is quantified in real time as it accumulates. For multiplex reactions involving more than one distinct probe, each of the probes can be labeled with a different distinguishable and detectable label.

The probes can be molecular beacons. Molecular beacons are single-stranded probes that form a stem-loop structure. A fluorophore can be, for example, covalently linked to one end of the stem and a quencher can be covalently linked to the other end of the stem forming a stem hybrid. When a molecular beacon hybridizes to a target nucleic acid sequence, the probe undergoes a conformational change that results in the dissociation of the stem hybrid and, thus the fluorophore and the quencher move away from each other, enabling the probe to fluoresce brightly. Molecular beacons can be labeled with differently colored fluorophores to detect different target sequences. Any of the probes described herein can be modified and utilized as molecular beacons.

Primer or probe sequences can be ranked according to specific hybridization parameters or metrics that assign a score value indicating their ability to anneal to viral strains under highly stringent conditions. Where a primer set is being scored, a “first” or “forward” primer is scored and the “second” or “reverse”-oriented primer sequences can be optimized similarly but with potentially additional parameters, followed by an optional evaluation for primer dimmers, for example, between the forward and reverse primers.

The scoring or ranking steps that are used in the methods of determining the primers and probes include, for example, the following parameters: a target sequence score for the target nucleic acid sequence(s), e.g., the PriMD® score; a mean conservation score for the target nucleic acid sequence(s); a mean coverage score for the target nucleic acid sequence(s); 100% conservation score of a portion (e.g., 5′ end, center, 3′ end) of the target nucleic acid sequence(s); a species score; a strain score; a subtype score; a serotype score; an associated disease score; a year score; a country of origin score; a duplicate score; a patent score; and a minimum qualifying score. Other parameters that are used include, for example, the number of mismatches, the number of critical mismatches (e.g., mismatches that result in the predicted failure of the sequence to anneal to a target sequence), the number of native strain sequences that contain critical mismatches, and predicted Tm values. The term “Tm” refers to the temperature at which a population of double-stranded nucleic acid molecules becomes half-dissociated into single strands. Methods for calculating the Tm of nucleic acids are known in the art (Berger and Kimmel (1987) Meth. Enzymol., Vol. 152: Guide To Molecular Cloning Techniques, San Diego: Academic Press, Inc. and Sambrook et al. (1989) Molecular Cloning: A Laboratory Manual, (2nd ed.) Vols. 1-3, Cold Spring Harbor Laboratory).

The resultant scores represent steps in determining nucleotide or whole target nucleic acid sequence preference, while tailoring the primer and/or probe sequences so that they hybridize to a plurality of target nucleic acid strains. The methods of determining the primers and probes also can comprise the step of allowing for one or more nucleotide changes when determining identity between the candidate primer and probe sequences and the target nucleic acid strain sequences, or their complements.

In another embodiment, the methods of determining the primers and probes comprise the steps of comparing the candidate primer and probe nucleic acid sequences to “exclusion nucleic acid sequences” and then rejecting those candidate nucleic acid sequences that share identity with the exclusion nucleic acid sequences. In another embodiment, the methods comprise the steps of comparing the candidate primer and probe nucleic acid sequences to “inclusion nucleic acid sequences” and then rejecting those candidate nucleic acid sequences that do not share identity with the inclusion nucleic acid sequences.

In other embodiments of the methods of determining the primers and probes, optimizing primers and probes comprises using a polymerase chain reaction (PCR) penalty score formula comprising at least one of a weighted sum of: primer Tm—optimal Tm; difference between primer Tms; amplicon length—minimum amplicon length; and distance between the primer and a TagMan® probe. The optimizing step also can comprise determining the ability of the candidate sequence to hybridize with the most target nucleic acid strain sequences (e.g., the most target organisms or genes). In another embodiment, the selecting or optimizing step comprises determining which sequences have mean conservation scores closest to 1, wherein a standard of deviation on the mean conservation scores is also compared.

In other embodiments, the methods further comprise the step of evaluating which target nucleic acid strain sequences are hybridized by an optimal forward primer and an optimal reverse primer, for example, by determining the number of base differences between target nucleic acid strain sequences in a database. For example, the evaluating step can comprise performing an in silico polymerase chain reaction, involving (1) rejecting the forward primer and/or reverse primer if it does not meet inclusion or exclusion criteria; (2) rejecting the forward primer and/or reverse primer if it does not amplify a medically valuable nucleic acid; (3) conducting a BLAST analysis to identify forward primer sequences and/or reverse primer sequences that overlap with a published and/or patented sequence; (4) and/or determining the secondary structure of the forward primer, reverse primer, and/or target. In an embodiment, the evaluating step includes evaluating whether the forward primer sequence, reverse primer sequence, and/or probe sequence hybridizes to sequences in the database other than the nucleic acid sequences that are representative of the target strains.

The present invention provides oligonucleotides that have preferred primer and probe qualities. These qualities are specific to the sequences of the optimized probes; however, one of ordinary skill in the art would recognize that other molecules with similar sequences could also be used. The oligonucleotides provided herein comprise a sequence that shares at least about 60-70% identity with a sequence described in Table 2. In addition, the sequences can be incorporated into longer sequences, provided they function to specifically anneal to and identify prostate cancer biomarkers and/or viral genes. In another embodiment, the invention provides a nucleic acid comprising a sequence that shares at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity with the sequences of Table 2 or complement thereof.

The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art. The primer and/or probe nucleic acid sequences of the invention are complementary to the target nucleic acid sequence. The probe and/or primer nucleic acid sequences of the invention are optimal for identifying numerous alleles or variants of a target nucleic acid, e.g., from PAP. In an embodiment, the nucleic acids of the invention are primers for the synthesis (e.g., amplification) of target nucleic acid strains and/or probes for identification, isolation, detection, quantitation or analysis of target nucleic acid variants, e.g., an amplified target nucleic acid variant that is amplified using the primers of the present invention.

The present oligonucleotides hybridize with more than one allele or variant (alleles and variants as determined by differences in their genomic sequence). The probes and primers provided herein can, for example, allow for the detection and quantitation of currently identified alleles or variants or a subset thereof. In addition, the primers and probes of the present invention, depending on the strain sequence(s), can allow for the detection and quantitation of previously unidentified alleles or variants. The methods of the invention provide for optimal primers and probes, and sets thereof, and combinations of sets thereof, which can hybridize with a larger number of target alleles or variants than available primers and probes.

In other aspects, the invention also provides vectors (e.g., plasmid, phage, expression), cell lines (e.g., mammalian, insect, yeast, bacterial), and kits comprising any of the sequences of the invention described herein. The invention further provides known or previously unknown target nucleic acid strain sequences that are identified, for example, using the methods of the invention. In an embodiment, the target nucleic acid strain sequence is an amplification product. In another embodiment, the target nucleic acid strain sequence is a native or synthetic nucleic acid. The primers, probes, target nucleic acid strain sequences, vectors, cell lines, and kits can have any number of uses, such as diagnostic, investigative, confirmatory, monitoring, predictive or prognostic.

Diagnostic kits that comprise one or more of the oligonucleotides described herein, which are useful for detecting RNAseL, PAP, Pim-1, Pim-2, Hepsin, PSMA, BK Virus or EBV in an individual and/or from a sample, are provided herein. An individual can be a human male, human female, human adult, human child, or human fetus. An individual can also be any mammal, reptile, avian, fish, or amphibian. Hence, an individual can be a primate, pig, horse, cattle, sheep, dog, rabbit, guinea pig, rodent, bird or fish. A sample includes blood, urine, serum, plasma, neoplastic or other tissue obtained from biopsies and samples specifically obtained from the prostate, cerebrospinal fluid, and the central nervous system.

A probe of the present invention can comprise a label such as, for example, a fluorescent label, a chemiluminescent label, a radioactive label, biotin, gold, dendrimers, aptamer, enzymes, proteins, quenchers and molecular motors. The probe may also be labeled with other similar detectable labels used in conjunction with probe technology as known by one of ordinary skill in the art. In an embodiment, the probe is a hydrolysis probe, such as, for example, a TaqMan® probe. In other embodiments, the probes of the invention are molecular beacons, any fluorescent probes, and probes that are replaced by any double stranded DNA binding dyes.

Oligonucleotides of the present invention do not only include primers that are useful for conducting the aforementioned amplification reactions, but also include oligonucleotides that are attached to a solid support, such as, for example, a microarray, multiwell plate, column, bead, glass slide, polymeric membrane, glass microfiber, plastic tubes, cellulose, and carbon nanostructures. Hence, detection of RNAseL, PAP, Pim-1, Pim-2, Hepsin, PSMA, BK Virus or EBV can be performed by exposing such an oligonucleotide-covered surface to a sample such that the binding of a complementary DNA sequence to a surface-attached oligonucleotide elicits a detectable signal or reaction.

Oligonucleotides of the present invention also include primers for isolating, quantitating and sequencing nucleic acid sequences derived from any identified or yet to be isolated and identified RNAseL, PAP, Pim-1, Pim-2, Hepsin, PSMA, BK Virus or EBV allele.

One embodiment of the invention uses solid support-based oligonucleotide hybridization methods to detect gene expression. Solid support-based methods suitable for practicing the present invention are widely known and are described (PCT application WO 95/11755; Huber et al., Anal. Biochem., 299:24, 2001; Meiyanto et al., Biotechniques, 31:406, 2001; Relogio et al., Nucleic Acids Res., 30:e51, 2002; the contents of which are incorporated herein by reference in their entirety). Any solid surface to which oligonucleotides can be bound, covalently or non-covalently, may be used. Such solid supports include, but are not limited to, filters, polyvinyl chloride dishes, silicon or glass based chips.

In certain embodiments, the nucleic acid molecule can be directly bound to the solid support or bound through a linker arm, which is typically positioned between the nucleic acid sequence and the solid support. A linker arm that increases the distance between the nucleic acid molecule and the substrate can increase hybridization efficiency. There are a number of ways to position a linker arm. In one common approach, the solid support is coated with a polymeric layer that provides linker arms with a plurality of reactive ends/sites. A common example of this type is glass slides coated with polylysine (U.S. Pat. No. 5,667,976, the contents of which are incorporated herein by reference in its entirety), which are commercially available. Alternatively, the linker arm can be synthesized as part of or conjugated to the nucleic acid molecule, and then this complex is bonded to the solid support. One approach, for example, takes advantage of the extremely high affinity biotin-streptavidin interaction. The streptavidin-biotinylated reaction is stable enough to withstand stringent washing conditions and is sufficiently stable that it is not cleaved by laser pulses used in some detection systems, such as matrix-assisted laser desorption/ionization time of flight (MALDI-TOF) mass spectrometry. Therefore, streptavidin can be covalently attached to a solid support, and a biotinylated nucleic acid molecule will bind to the streptavidin-coated surface. In one version of this method, an amino-coated silicon wafer is reacted with the n-hydroxysuccinimido-ester of biotin and complexed with streptavidin. Biotinylated oligonucleotides are bound to the surface at a concentration of about 20 fmol DNA per mm².

One can alternatively directly bind DNA to the support using carbodiimides, for example. In one such method, the support is coated with hydrazide groups, and then treated with carbodiimide. Carboxy-modified nucleic acid molecules are then coupled to the treated support. Epoxide-based chemistries are also being employed with amine modified oligonucleotides. Other chemistries for coupling nucleic acid molecules to solid substrates are known to those of one of ordinary skill in the art.

The nucleic acid molecules, e.g., the primers and probes of the present invention, must be delivered to the substrate material, which is suspected of containing or is being tested for the presence and amount of RNAseL, PAP, Pim-1, Pim-2, Hepsin, PSMA, BK Virus or EBV. Because of the miniaturization of the arrays, delivery techniques must be capable of positioning very small amounts of liquids in very small regions, very close to one another and amenable to automation. Several techniques and devices are available to achieve such delivery. Among these are mechanical mechanisms (e.g., arrayers from GeneticMicroSystems, MA, USA) and ink jet technology. Very fine pipets can also be used.

Other formats are also suitable within the context of this invention. For example, a 96-well format with fixation of the nucleic acids to a nitrocellulose or nylon membrane can also be employed.

After the nucleic acid molecules have been bound to the solid support, it is often useful to block reactive sites on the solid support that are not consumed in binding to the nucleic acid molecule. In the absence of the blocking step, excess primers and/or probes can, to some extent, bind directly to the solid support itself, giving rise to non-specific binding. Non-specific binding can sometimes hinder the ability to detect low levels of specific binding. A variety of effective blocking agents (e.g., milk powder, serum albumin or other proteins with free amine groups, polyvinylpyrrolidine) can be used and others are known to those skilled in the art (U.S. Pat. No. 5,994,065, the contents of which are incorporated herein by reference in their entirety). The choice depends at least in part upon the binding chemistry.

One embodiment uses oligonucleotide arrays, e.g., microarrays that can be used to simultaneously observe the expression of RNAseL, PAP, Pim-1, Pim-2, Hepsin, PSMA, BK Virus or EBV. Oligonucleotide arrays comprise two or more oligonucleotide probes provided on a solid support, wherein each probe occupies a unique location on the support. The location of each probe can be predetermined, such that detection of a detectable signal at a given location is indicative of hybridization to an oligonucleotide probe of a known identity. Each predetermined location can contain more than one molecule of a probe, but each molecule within the predetermined location has an identical sequence. Such predetermined locations are termed features. There can be, for example, from 2, 10, 100, 1,000, 2,000 or 5,000 or more of such features on a single solid support. In one embodiment, each oligonucleotide is located at a unique position on an array at least 2, at least 3, at least 4, at least 5, at least 6, or at least 10 times.

Oligonucleotide probe arrays for detecting gene expression can be made and used according to conventional techniques described (Lockhart et al., Nat. Biotech., 14:1675-1680, 1996; McGall et al., Proc. Natl. Acad. Sci. USA, 93:13555, 1996; Hughes et al., Nat. Biotechnol., 19:342, 2001). A variety of oligonucleotide array designs are suitable for the practice of this invention.

Generally, a detectable molecule, also referred to herein as a label, can be incorporated or added to an array's probe nucleic acid sequences. Many types of molecules can be used within the context of this invention. Such molecules include, but are not limited to, fluorochromes, chemiluminescent molecules, chromogenic molecules, radioactive molecules, mass spectrometry tags, proteins, and the like. Other labels will be readily apparent to one skilled in the art.

Oligonucleotide probes used in the methods of the present invention, including microarray techniques, can be generated using PCR. PCR primers used in generating the probes are chosen, for example, based on the sequences of Table 2. In one embodiment, oligonucleotide control probes also are used. Exemplary control probes can fall into at least one of three categories referred to herein as (1) normalization controls, (2) expression level controls and (3) negative controls. In microarray methods, one or more of these control probes can be provided on the array with the inventive cell cycle gene-related oligonucleotides.

Normalization controls correct for dye biases, tissue biases, dust, slide irregularities, malformed slide spots, etc. Normalization controls are oligonucleotide or other nucleic acid probes that are complementary to labeled reference oligonucleotides or other nucleic acid sequences that are added to the nucleic acid sample to be screened. The signals obtained from the normalization controls, after hybridization, provide a control for variations in hybridization conditions, label intensity, reading efficiency and other factors that can cause the signal of a perfect hybridization to vary between arrays. The normalization controls also allow for the semi-quantification of the signals from other features on the microarray. In one embodiment, signals (e.g., fluorescence intensity or radioactivity) read from all other probes used in the method are divided by the signal from the control probes, thereby normalizing the measurements.

Virtually any probe can serve as a normalization control. Hybridization efficiency varies, however, with base composition and probe length. Preferred normalization probes are selected to reflect the average length of the other probes being used, but they also can be selected to cover a range of lengths. Further, the normalization control(s) can be selected to reflect the average base composition of the other probe(s) being used. In one embodiment, only one or a few normalization probes are used, and they are selected such that they hybridize well (i.e., without forming secondary structures) and do not match any test probes. In one embodiment, the normalization controls are mammalian genes.

“Negative control” probes are not complementary to any of the test oligonucleotides (i.e., the prostate cancer biomarker oligonucleotides), normalization controls, or expression controls. In one embodiment, the negative control is a mammalian gene which is not complementary to any other sequence in the sample.

The terms “background” and “background signal intensity” refer to hybridization signals resulting from non-specific binding or other interactions between the labeled target nucleic acids (e.g., mRNA present in the biological sample) and components of the oligonucleotide array. Background signals also can be produced by intrinsic fluorescence of the array components themselves. A single background signal can be calculated for the entire array, or a different background signal can be calculated for each target nucleic acid. In one embodiment, background is calculated as the average hybridization signal intensity for the lowest 5 to 10 percent of the oligonucleotide probes being used, or, where a different background signal is calculated for each target gene, for the lowest 5 to 10 percent of the probes for each gene. Where the oligonucleotide probes corresponding to a particular prostate cancer biomarker or viral gene target hybridize well and, hence, appear to bind specifically to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample). In microarray methods, background can be calculated as the average signal intensity produced by regions of the array that lack any oligonucleotides probes at all.

In an alternative embodiment, the nucleic acid molecules are directly or indirectly coupled to an enzyme. Following hybridization, a chromogenic substrate is applied and the colored product is detected by a camera, such as a charge-coupled camera. Examples of such enzymes include alkaline phosphatase, horseradish peroxidase and the like. The invention also provides methods of labeling nucleic acid molecules with cleavable mass spectrometry tags (CMST; U.S. Patent Application No. 60/279,890). After an assay is complete, and the uniquely CMST-labeled probes are distributed across the array, a laser beam is sequentially directed to each member of the array. The light from the laser beam both cleaves the unique tag from the tag-nucleic acid molecule conjugate and volatilizes it. The volatilized tag is directed into a mass spectrometer. Based on the mass spectrum of the tag and knowledge of how the tagged nucleotides were prepared, one can unambiguously identify the nucleic acid molecules to which the tag was attached (WO 9905319, the entire contents of which are hereby incorporated by reference).

The nucleic acids, primers and probes of the present invention can be labeled readily by any of a variety of techniques. When the diversity panel is generated by amplification, the nucleic acids can be labeled during the reaction by incorporation of a labeled dNTP or use of labeled amplification primer. If the amplification primers include a promoter for an RNA polymerase, a post-reaction labeling can be achieved by synthesizing RNA in the presence of labeled NTPs. Amplified fragments that were unlabeled during amplification or unamplified nucleic acid molecules can be labeled by one of a number of end labeling techniques or by a transcription method, such as nick-translation, random-primed DNA synthesis. Details of these methods are known to one of ordinary skill in the art and are set out in methodology books. Other types of labeling reactions are performed by denaturation of the nucleic acid molecules in the presence of a DNA-binding molecule, such as RecA, and subsequent hybridization under conditions that favor the formation of a stable RecA-incorporated DNA complex.

In another embodiment, PCR-based methods are used to detect gene expression. These methods include reverse-transcriptase-mediated polymerase chain reaction (RT-PCR) including real-time and endpoint quantitative reverse-transcriptase-mediated polymerase chain reaction (Q-RTPCR). These methods are well known in the art. For example, methods of quantitative PCR can be carried out using kits and methods that are commercially available from, for example, Applied BioSystems and Stratagene®. See also Kochanowski, Quantitative PCR Protocols (Humana Press, 1999); Innis et al., supra.; Vandesompele et al., Genome Biol., 3:RESEARCH0034, 2002; Stein, Cell Mol. Life Sci. 59:1235, 2002.

The forward and reverse amplification primers and internal hybridization probe is designed to hybridize specifically and uniquely with one nucleotide sequence derived from the transcript of a target gene. In one embodiment, the selection criteria for primer and probe sequences incorporates constraints regarding nucleotide content and size to accommodate TaqMan® requirements. SYBR Green® can be used as a probe-less Q-RTPCR alternative to the TaqMan®-type assay, discussed above (ABI Prism® 7900 Sequence Detection System User Guide Applied Biosystems, chap. 1-8, App. A-F. (2002)). This device measures changes in fluorescence emission intensity during PCR amplification. The measurement is done in “real time,” that is, as the amplification product accumulates in the reaction. Other methods can be used to measure changes in fluorescence resulting from probe digestion. For example, fluorescence polarization can distinguish between large and small molecules based on molecular tumbling (U.S. Pat. No. 5,593,867).

The primers and probes of the present invention may anneal to or hybridize to various prostate cancer biomarker or viral genetic material or genetic material derived therefrom, such as RNA, DNA, cDNA, or a PCR product.

A “sample” that is tested for the presence of RNAseL, PAP, Pim-1, Pim-2, Hepsin, PSMA, BK Virus or EBV includes, but is not limited to a tissue sample, such as, for example, blood, urine, serum, plasma, neoplastic or other tissue obtained from biopsies and samples specifically obtained from the prostate, cerebrospinal fluid, and the central nervous system. In a particular embodiment, the sample is from a human, is non-human in origin, or is derived from an inanimate object. The tissue sample may be fresh, fixed, preserved, or frozen.

The target nucleic acid strain that is amplified may be RNA or DNA or a modification thereof If the target nucleic acid strain is RNA, the RNA can be reverse transcribed into cDNA using a reverse transcriptase primer and reverse transcriptase. Thus, the amplifying step can comprise isothermal or non-isothermal reactions, such as polymerase chain reaction, Scorpion® primers, molecular beacons, SimpleProbes®, HyBeacons®, cycling probe technology, Invader Assay, self-sustained sequence replication, nucleic acid sequence-based amplification, ramification amplifying method, hybridization signal amplification method, rolling circle amplification, multiple displacement amplification, thermophilic strand displacement amplification, transcription-mediated amplification, ligase chain reaction, signal mediated amplification of RNA, split promoter amplification, Q-Beta replicase, isothermal chain reaction, one cut event amplification, loop-mediated isothermal amplification, molecular inversion probes, ampliprobe, headloop DNA amplification, and ligation activated transcription. The amplifying step can be conducted on a solid support, such as a multiwell plate, array, column, bead, glass slide, polymeric membrane, glass microfiber, plastic tubes, cellulose, and carbon nanostructures. The amplifying step also comprises in situ hybridization. The detecting step can comprise gel electrophoresis, fluorescence resonant energy transfer, or hybridization to a labeled probe, such as a probe labeled with biotin, at least one fluorescent moiety, an antigen, a molecular weight tag, and a modifier of probe Tm. The detection step can also comprise the incorporation of a label (e.g. fluorescent or radioactive) during an extension reaction. The detecting step comprises measuring fluorescence, mass, charge, and/or chemiluminescence.

The target nucleic acid strain may not need amplification and may be RNA or DNA or a modification thereof If amplification is not necessary, the target nucleic acid strain can be denatured to enable hybridization of a probe to the target nucleic acid sequence.

Hybridization may be detected in a variety of ways and with a variety of equipment. In general, the methods can be categorized as those that rely upon detectable molecules incorporated into the diversity panels and those that rely upon measurable properties of double-stranded nucleic acids (e.g., hybridized nucleic acids) that distinguish them from single-stranded nucleic acids (e.g., unhybridized nucleic acids). The latter category of methods includes intercalation of dyes, such as, for example, ethidium bromide, into double-stranded nucleic acids, differential absorbance properties of double and single stranded nucleic acids, binding of proteins that preferentially bind double-stranded nucleic acids, and the like.

### EXEMPLIFICATION

### Example 1

### Scoring a Set of Predicted Annealing Oligonucleotides

Each of the sets of primers and probes selected is ranked by a combination of methods as individual primers and probes and as a primer/probe set. This involves one or more methods of ranking (e.g., joint ranking, hierarchical ranking , and serial ranking) where sets of primers and probes are eliminated or included based on any combination of the following criteria, and a weighted ranking again based on any combination of the following criteria, for example: (A) Percentage Identity to Target Strains; (B) Conservation Score; (C) Coverage Score; (D) Strain/Subtype/Serotype Score; (E) Associated Disease Score; (F) Duplicates Sequences Score; (G) Year and Country of Origin Score; (H) Patent Score, and (I) Epidemiology Score.

### A. Percentage Identity

A percentage identity score is based upon the number of target nucleic acid strain (e.g., native) sequences that can hybridize with perfect conservation (the sequences are perfectly complimentary) to each primer or probe of a primer set and probe set. If the score is less than 100%, the program ranks additional primer set and probe sets that are not perfectly conserved. This is a hierarchical scale for percent identity starting with perfect complimentarity, then one base degeneracy through to the number of degenerate bases that would provide the score closest to 100%. The position of these degenerate bases would then be ranked. The methods for calculating the conservation is described under section B.

(i) Individual Base Conservation Score

A set of conservation scores is generated for each nucleotide base in the consensus sequence and these scores represent how many of the target nucleic acid strains sequences have a particular base at this position. For example, a score of 0.95 for a nucleotide with an adenosine, and 0.05 for a nucleotide with a cytidine means that 95% of the native sequences have an A at that position and 5% have a C at that position. A perfectly conserved base position is one where all the target nucleic acid strain sequences have the same base (either an A, C, G, or T/U) at that position. If there is an equal number of bases (e.g., 50% A & 50% T) at a position, it is identified with an N.

(ii) Candidate Primer/Probe Sequence Conservation

An overall conservation score is generated for each candidate primer or probe sequence that represents how many of the target nucleic acid strain sequences will hybridize to the primers or probes. A candidate sequence that is perfectly complimentary to all the target nucleic acid strain sequences will have a score of 1.0 and rank the highest. For example, illustrated below in Table 1 are three different 10-base candidate probe sequences that are targeted to different regions of a consensus target nucleic acid strain sequence. Each candidate probe sequence is compared to a total of 10 native sequences.

```
TABLE 1
SEQ ID NO: 62
#1. A A A C A C G T G C
        0.7 1.0 1.0 1.0 1.0 1.0 1.0 1.0 1.0 1.0
→Number of target nucleic acid strain sequences
that are perfectly complimentary-7. Three out of
the ten sequences do not have an A at position 1.
SEQ ID NO: 63
#2. C C T T G T T C C A
        1.0 0.9 1.0 0.9 0.9 1.0 1.0 1.0 1.0 1.0
→Number of target nucleic acid strain sequences
that are perfectly complimentary-7, 8, or 9. At
least one target nucleic acid strain does not
have a C at position 2, T at position 4, or G
at position 5. These differences may all be on
one target nucleic acid strain molecule or may
be on two or three separate molecules.
SEQ ID NO: 64
#3. C A G G G A C G A T
        1.0 1.0 1.0 1.0 1.0 0.9 0.8 1.0 1.0 1.0
→Number of target nucleic acid strain sequences
that are perfectly complimentary-7 or 8. At
least one target nucleic acid strain does not
have an A at position 6 and at least two target
nucleic acid strain do not have a C at position
7. These differences may all be on one target
nucleic acid strain molecule or may be on two
separate molecules.
```

A simple arithmetic mean for each candidate sequence would generate the same value of 0.97. The number of target nucleic acid strain sequences identified by each candidate probe sequence, however, can be very different. Sequence #1 can only identify 7 native sequences because of the 0.7 (out of 1.0) score by the first base—A. Sequence #2 has three bases each with a score of 0.9; each of these could represent a different or shared target nucleic acid strain sequence. Consequently, Sequence #2 can identify 7, 8 or 9 target nucleic acid strain sequences. Similarly, Sequence #3 can identify 7 or 8 of the target nucleic acid strain sequences. Sequence #2 would, therefore, be the best choice if all the three bases with a score of 0.9 represented the same 9 target nucleic acid strain sequences.

(iii) Overall Conservation Score of the Primer and Probe Set—Percent Identity

The same method described in (ii) when applied to the complete primer set and probe set will generate the percent identity for the set (see A above). For example, using the same sequences illustrated above, if Sequences #1 and #2 are primers and Sequence #3 is a probe, then the percent identity for the target can be calculated from how many of the target nucleic acid strain sequences are identified with perfect complimentarity by all three primer/probe sequences. The percent identity could be no better than 0.7 (7 out of 10 target nucleic acid strain sequences) but as little as 0.1 if each of the degenerate bases reflects a different target nucleic acid strain sequence. Again, an arithmetic mean of these three sequences would be 0.97. As none of the above examples were able to capture all the target nucleic acid strain sequences because of the degeneracy (scores of less than 1.0), the ranking system takes into account that a certain amount of degeneracy can be tolerated under normal hybridization conditions, for example, during a polymerase chain reaction. The ranking of these degeneracies is described in (iv) below.

An in silico evaluation determines how many native sequences (e.g., original sequences submitted to public databases) are identified by a given candidate primer/probe set. The ideal candidate primer/probe set is one that can perform PCR and the sequences are perfectly complimentary to all the known native sequences that were used to generate the consensus sequence. If there is no such candidate, then the sets are ranked according to how many degenerate bases can be accepted and still hybridize to only the target sequence during the PCR and yet identify all the native sequences.

The hybridization conditions, for TagMan® as an example, are: 10-50 mM Tris-HCl pH 8.3, 50 mM KCl, 0.1-0.2% Triton® X-100 or 0.1% Tween®, 1-5 mM MgCl₂. The hybridization is performed at 58-60° C. for the primers and 68-70° C. for the probe. The in silico PCR identifies native sequences that are not amplifiable using the candidate primers and probe set. The rules can be as simple as counting the number of degenerate bases to more sophisticated approaches based on exploiting the PCR criteria used by the PriMD® software. Each target nucleic acid strain sequence has a value or weight (see Score assignment above). If the failed target nucleic acid strain sequence is medically valuable, the primer/probe set is rejected. This in silico analysis provides a degree of confidence for a given genotype and is important when new sequences are added to the databases. New target nucleic acid strain sequences are automatically entered into both the “include” and “exclude” categories. Published primer and probes will also be ranked by the PriMD software.

(iv) Position (5′ to 3′) of The Base Conservation Score

In an embodiment, primers do not have bases in the terminal five positions at the 3′ end with a score less than 1. This is one of the last parameters to be relaxed if the method fails to select any candidate sequences. The next best candidate having a perfectly conserved primer would be one where the poorer conserved positions are limited to the terminal bases at the 5′ end. The closer the poorer conserved position is to the 5′ end, the better the score. For probes, the position criteria are different. For example, with a TaqMan® probe, the most destabilizing effect occurs in the center of the probe. The 5′ end of the probe is also important as this contains the reporter molecule that must be cleaved, following hybridization to the target, by the polymerase to generate a sequence-specific signal. The 3′ end is less critical. Therefore, a sequence with a perfectly conserved middle region will have the higher score. The remaining ends of the probe are ranked in a similar fashion to the 5′ end of the primer. Thus, the next best candidate to a perfectly conserved TaqMan® probe would be one where the poorer conserved positions are limited to the terminal bases at either the 5′ or 3′ ends. The hierarchical scoring will select primers with only one degeneracy first, then primers with two degeneracies next and so on. The relative position of each degeneracy will then be ranked favoring those that are closest to the 5′ end of the primers and those closest to the 3′ end of the TagMan® probe. If there are two or more degenerate bases in a primer and probe set, the ranking will initially select the sets where the degeneracies occur on different sequences.

### B. Coverage Score

The total number of aligned sequences is considered under a coverage score. A value is assigned to each position based on how many times that position has been reported or sequenced. Alternatively, coverage can be defined as how representative the sequences are of the known strains, subtypes etc., or their relevance to a certain diseases. For example, the target nucleic acid strain sequences for a particular gene may be very well conserved and show complete coverage but certain strains are not represented in those sequences.

A sequence is included if it aligns with any part of the consensus sequence, which is usually a whole gene or a functional unit, or has been described as being a representative of this gene. Even though a base position is perfectly conserved it may only represent a fraction of the total number of sequences (for example, if there are very few sequences). For example, region A of a gene shows a 100% conservation from 20 sequence entries while region B in the same gene shows a 98% conservation but from 200 sequence entries. There is a relationship between conservation and coverage if the sequence shows some persistent variability. As more sequences are aligned, the conservation score falls, but this effect is lessened as the number of sequences gets larger. Unless the number of sequences is very small (e.g., under 10) the value of the coverage score is small compared to that of the conservation score. To obtain the best consensus sequence, artificial spaces are allowed to be introduced. Such spaces are not considered in the coverage score.

### C. Strain/Subtype/Serotype Score

A value is assigned to each strain or subtype or serotype based upon its relevance to a disease. For example, strains of BK Virus that are linked to high frequencies of infection will have a higher score than strains that are generally regarded as benign. The score is based upon sufficient evidence to automatically associate a particular strain with a disease.

### D. Associated Disease Score

The associated disease score pertains to strains that are not known to be associated with a particular disease (to differentiate from D above). Here, a value is assigned only if the submitted sequence is directly linked to the disease and that disease is pertinent to the assay.

### E. Duplicate Sequences Score

If a particular sequence has been sequenced more than once it will have an effect on representation, for example, a strain that is represented by 12 entries in GenBank of which six are identical and the other six are unique. Unless the identical sequences can be assigned to different strains/subtypes (usually by sequencing other genes or by immunology methods) they will be excluded from the scoring.

### F. Year and Country of Origin Score

The year and country of origin scores are important in terms of the age of the human population and the need to provide a product for a global market. For example, strains identified or collected many years ago may not be relevant today. Furthermore, it is probably difficult to obtain samples that contain these older strains. Certain divergent strains from more obscure countries or sources may also be less relevant to the locations that will likely perform clinical tests, or may be more important for certain countries (e.g., North America, Europe, or Asia).

### G. Patent Score

Candidate target strain sequences published in patents are searched electronically and annotated such that patented regions are excluded. Alternatively, candidate sequences are checked against a patented sequence database.

### H. Minimum Qualifying Score

The minimum qualifying score is determined by expanding the number of allowed mismatches in each set of candidate primers and probes until all possible native sequences are represented (e.g., has a qualifying hit).

### I. Other

A score is given to based on other parameters, such as relevance to certain patients (e.g., pediatrics, immunocompromised) or certain therapies (e.g., target those strains that respond to treatment) or epidemiology. The prevalence of an organism/strain and the number of times it has been tested for in the community can add value to the selection of the candidate sequences. If a particular strain is more commonly tested then selection of it would be more likely. Strain identification can be used to select better vaccines.

### Example 2

### Primer/Probe Evaluation

Once the candidate primers and probes have received their scores and have been ranked, they are evaluated using any of a number of methods of the invention, such as BLAST analysis and secondary structure analysis.

### A. BLAST Analysis

The candidate primer/probe sets are submitted to BLAST analysis to check for possible overlap with any published sequences that might be missed by the Include/Exclude function. It also provides a useful summary.

### B. Secondary Structure

The methods of the present invention include analysis of nucleic acid secondary structure. This includes the structures of the primers and/or probes, as well as their intended target strain sequences. The methods and software of the invention predict the optimal temperatures for annealing, but assumes that the target (e.g., RNA or DNA) does not have any significant secondary structure. For example, if the starting material is RNA, the first stage is the creation of a complimentary strand of DNA (cDNA) using a specific primer. This is usually performed at temperatures where the RNA template can have significant secondary structure thereby preventing the annealing of the primer. Similarly, after denaturation of a double stranded DNA target (for example, an amplicon after PCR), the binding of the probe is dependent on there being no major secondary structure in the amplicon.

The methods of the invention can either use this information as a criteria for selecting primers and probes or evaluate any secondary structure of a selected sequence, for example, by cutting and pasting candidate primer or probe sequences into a commercial internet link that uses software dedicated to analyzing secondary structure, such as, for example, MFOLD (Zuker et al. (1999) Algorithms and Thermodynamics for RNA Secondary Structure Prediction: A Practical Guide in RNA Biochemistry and Biotechnology, J. Barciszewski and B. F. C. Clark, eds., NATO ASI Series, Kluwer Academic Publishers).

### C. Evaluating the Primer and Probe Sequences

The methods and software of the invention may also analyze any nucleic acid sequence to determine its suitability in a nucleic acid amplification-based assay. For example, it can accept a competitor's primer set and determine the following information: (1) How it compares to the primers of the invention (e.g., overall rank, PCR and conservation ranking, etc.); (2) How it aligns to the excluded libraries (e.g., assessing cross-hybridization)—also used to compare primer and probe sets to newly published sequences; and (3) If the sequence has been previously published. This step requires keeping a database of sequences published in scientific journals, posters, and other presentations.

### Example 3

### Multiplexing

The Exclude/Include capability is ideally suited for designing multiplex reactions. The parameters for designing multiple primer and probe sets adhere to a more stringent set of parameters than those used for the initial Exclude/Include function. Each set of primers and probes, together with the resulting amplicon, is screened against the other sets that constitute the multiplex reaction. As new targets are accepted, their sequences are automatically added to the Exclude category.

The database is designed to interrogate the online databases to determine and acquire, if necessary, any new sequences relevant to the targets. These sequences are evaluated against the optimal primer/probe set. If they represent a new genotype or strain, then a multiple sequence alignment may be required.

### Example 4

### Sequences Identified for Detecting RNAseL, PAP, Pim-1, Pim-2, Hepsin or PSMA

The set of primers and probes were then scored according to the methods described herein to identify the optimized primers and probes of Table 2. It should be noted that the primers, as they are sequences that anneal to RNAseL, PAP, Pim-1, Pim-2, Hepsin or PSMA, can also be used as probes either in the presence or absence of amplification of a sample.

TABLE 2
Optimized RNAse L, PAP, Pim-1, Pim-2, Hepsin
and PSMA Primers and Probes.

| | | | |
|---|---|---|---|
| Group | Forward Primer | Probe | Reverse Primer |

PAP

| | | | |
|---|---|---|---|
| 1 | SEQ ID NO: 1 | SEQ ID NO: 2 | SEQ ID NO: 3 |
| | GAAGTCCCATTGAC | CATAAAGGAATCCTC | TGGGTGAGTTGGC |
| | ACCTTTCC | ATGGCCACAA | CAAATCC |
| 2 | SEQ ID NO: 4 | SEQ ID NO: 2 | SEQ ID NO: 3 |
| | TTGACACCTTTCCC | CATAAAGGAATCCTC | TGGGTGAGTTGGC |
| | ACTGACC | ATGGCCACAA | CAAATCC |
| 3 | SEQ ID NO: 5 | SEQ ID NO: 6 | SEQ ID NO: 3 |
| | GAAGTCCCATTGAC | TGACCCCATAAAGGA | TGGGTGAGTTGGC |
| | ACCTTTCCCA | ATCCTCATGGCCAC | CAAATCC |
| 4 | SEQ ID NO: 7 | SEQ ID NO: 8 | SEQ ID NO: 3 |
| | CCGAAGTCCCATTG | CTTTCCCACTGACCC | TGGGTGAGTTGGC |
| | ACAC | CATAAAGGAATCCTC | CAAATCC |
| | | ATG | |

PIM-1

| | | | |
|---|---|---|---|
| 5 | SEQ ID NO: 9 | SEQ ID NO: 10 | SEQ ID NO: 11 |
| | GAGAAGGAGCCCC | TAGACCGAGCCGAAG | GGCAAGTTGTCGG |
| | TGGAG | CCGCC | AGACG |
| 6 | SEQ ID NO: 9 | SEQ ID NO: 12 | SEQ ID NO: 11 |
| | GAGAAGGAGCCCC | CTTCGGCTCGGTCTA | GGCAAGTTGTCGG |
| | TGGAG | CTCAGGCATC | AGACG |
| 7 | SEQ ID NO: 9 | SEQ ID NO: 13 | SEQ ID NO: 11 |
| | GAGAAGGAGCCCC | TGCCTGAGTAGACCG | GGCAAGTTGTCGG |
| | TGGAG | AGCCGAAGC | AGACG |
| 8 | SEQ ID NO: 9 | SEQ ID NO: 14 | SEQ ID NO: 11 |
| | GAGAAGGAGCCCC | CGGTCTACTCAGGCA | GGCAAGTTGTCGG |
| | TGGAG | TCCG | AGACG |
| 9 | SEQ ID NO: 9 | SEQ ID NO: 15 | SEQ ID NO: 11 |
| | GAGAAGGAGCCCC | CAGTACCAGGTGGGC | GGCAAGTTGTCGG |
| | TGGAG | CCGCT | AGACG |
| 10 | SEQ ID NO: 9 | SEQ ID NO: 16 | SEQ ID NO: 11 |
| | GAGAAGGAGCCCC | AGCGGCGGCTTCGGC | GGCAAGTTGTCGG |
| | TGGAG | TC | AGACG |
| 11 | SEQ ID NO: 9 | SEQ ID NO: 17 | SEQ ID NO: 11 |
| | GAGAAGGAGCCCC | AGCCGAAGCCGCCGC | GGCAAGTTGTCGG |
| | TGGAG | | AGACG |
| 12 | SEQ ID NO: 9 | SEQ ID NO: 18 | SEQ ID NO: 11 |
| | GAGAAGGAGCCCC | CGGGCCCACCTGGTA | GGCAAGTTGTCGG |
| | TGGAG | CTGC | AGACG |

PIM-2

| | | | |
|---|---|---|---|
| 13 | SEQ ID NO: 19 | SEQ ID NO: 21 | SEQ ID NO: 20 |
| | GCGCGGCGAATCT | CACCAGTTTCTCTGC | GGCCCCTGTAGAG |
| | CAAC | TTTCCACCCTGG | GCTT |
| 14 | SEQ ID NO: 19 | SEQ ID NO: 22 | SEQ ID NO: 20 |
| | GCGCGGCGAATCT | TCTGCTTTCCACCCT | GGCCCCTGTAGAG |
| | CAAC | GGCGCC | GCTT |
| 15 | SEQ ID NO: 19 | SEQ ID NO: 23 | SEQ ID NO: 20 |
| | GCGCGGCGAATCT | TTCCACCCTGGCGCC | GGCCCCTGTAGAG |
| | CAAC | CC | GCTT |
| 16 | SEQ ID NO: 19 | SEQ ID NO: 24 | SEQ ID NO: 20 |
| | GCGCGGCGAATCT | CACTGAACCCGCTAA | GGCCCCTGTAGAG |
| | CAAC | GCCCGCA | GCTT |
| 17 | SEQ ID NO: 19 | SEQ ID NO: 25 | SEQ ID NO: 20 |
| | GCGCGGCGAATCT | CTGCGCAGATTGAGC | GGCCCCTGTAGAG |
| | CAAC | CCACTGAAC | GCTT |
| 18 | SEQ ID NO: 19 | SEQ ID NO: 26 | SEQ ID NO: 20 |
| | GCGCGGCGAATCT | ACCCGCTAAGCCCGC | GGCCCCTGTAGAG |
| | CAAC | A | GCTT |
| 19 | SEQ ID NO: 19 | SEQ ID NO: 27 | SEQ ID NO: 20 |
| | GCGCGGCGAATCT | AGATTGAGCCCACTG | GGCCCCTGTAGAG |
| | CAAC | AACCCGCTAAG | GCTT |

HEPSIN

| | | | |
|---|---|---|---|
| 20 | SEQ ID NO: 28 | SEQ ID NO: 29 | SEQ ID NO: 30 |
| | GGACGCTCGGCTC | CAGCAGCCGCCACGT | GAAGCCCATCTCC |
| | ATG | CCC | TCGCA |
| 21 | SEQ ID NO: 31 | SEQ ID NO: 32 | SEQ ID NO: 30 |
| | GGACGCTCGGCTC | CCCTTCCGTCTTGTC | GAAGCCCATCTCC |
| | A | AAAGACCA | TCGCA |
| 22 | SEQ ID NO: 28 | SEQ ID NO: 33 | SEQ ID NO: 30 |
| | GGACGCTCGGCTC | CGGAAGGGACGTGGC | GAAGCCCATCTCC |
| | ATG | GGC | TCGCA |
| 23 | SEQ ID NO: 34 | SEQ ID NO: 35 | SEQ ID NO: 36 |
| | CTCGGCTCATGGTC | AGCTGAGTCCGGCTA | CTGAGGAAGCCCA |
| | TTTGACAA | CCCTGG | TCTCC |
| 24 | SEQ ID NO: 28 | SEQ ID NO: 37 | SEQ ID NO: 30 |
| | GGACGCTCGGCTC | CCACGTCCCTTCCGT | GAAGCCCATCTCC |
| | ATG | CTTGTCAAAGAC | TCGCA |
| 25 | SEQ ID NO: 34 | SEQ ID NO: 38 | SEQ ID NO: 36 |
| | CTCGGCTCATGGTC | CGTTGGAGCGCGAGG | CTGAGGAAGCCCA |
| | TTTGACAA | AGC | TCTCC |
| 26 | SEQ ID NO: 39 | SEQ ID NO: 40 | SEQ ID NO: 30 |
| | GACGCTCGGCTCAT | TTTGACAAGACGGAA | GAAGCCCATCTCC |
| | GG | GGGACGTGGC | TCGCA |
| 27 | SEQ ID NO: 34 | SEQ ID NO: 41 | SEQ ID NO: 30 |
| | CTCGGCTCATGGTC | TCCGGCTACCCTGGC | GAAGCCCATCTCC |
| | TTTGACAA | GTTGGA | TCGCA |

PSMA

| | | | |
|---|---|---|---|
| 28 | SEQ ID NO: 42 | SEQ ID NO: 43 | SEQ ID NO: 44 |
| | GAAACCGACTCGG | CGCCCCAGCGCACAG | AGCCGAGGAGAAA |
| | CTGTG | | GAAGCCA |
| 29 | SEQ ID NO: 42 | SEQ ID NO: 45 | SEQ ID NO: 44 |
| | GAAACCGACTCGG | CAGCACCAGCGCCCC | AGCCGAGGAGAAA |
| | CTGTG | AGC | GAAGCCA |
| 30 | SEQ ID NO: 42 | SEQ ID NO: 46 | SEQ ID NO: 47 |
| | GAAACCGACTCGG | TGGTGCTGGCGGGTG | GGAAGCCGAGGAG |
| | CTGTG | GC | AAAGA |
| 31 | SEQ ID NO: 42 | SEQ ID NO: 48 | SEQ ID NO: 49 |
| | GAAACCGACTCGG | CGCCCCAGCGCACAG | CACCCGCCAGCAC |
| | CTGTG | | CA |
| 32 | SEQ ID NO: 42 | SEQ ID NO: 50 | SEQ ID NO: 51 |
| | GAAACCGACTCGG | CCACCCGCCAGCACC | GGAAGCCGAGGAG |
| | CTGTG | AG | AAAGAAG |

RNAse L

| | | | |
|---|---|---|---|
| 33 | SEQ ID NO: 52 | SEQ ID NO: 53 | SEQ ID NO: 54 |
| | TTGGACGGCTGGT | CATGTTCCCCAGGAT | TCCCAAGTCCAAA |
| | CCTCTAT | GGAAGAGACGATGAA | AGAAGGGATG |
| | | TGA | |
| 34 | SEQ ID NO: 52 | SEQ ID NO: 55 | SEQ ID NO: 54 |
| | TTGGACGGCTGGT | CAAAGTAATGAAGAG | TCCCAAGTCCAAA |
| | CCTCTAT | GTGGTTCAACTTTCT | AGAAGGGATG |
| | | CCAGA | |
| 35 | SEQ ID NO: 52 | SEQ ID NO: 56 | SEQ ID NO: 54 |
| | TTGGACGGCTGGT | CTGAAAGCTCAAAGT | TCCCAAGTCCAAA |
| | CCTCTAT | AATGAAGAGGTGGTT | AGAAGGGATG |
| | | CAACT | |
| 36 | SEQ ID NO: 52 | SEQ ID NO: 57 | SEQ ID NO: 54 |
| | TTGGACGGCTGGT | AGGTGGTTCAACTTT | TCCCAAGTCCAAA |
| | CCTCTAT | CTCCAGATGAGGAAA | AGAAGGGATG |
| | | CTAAG | |
| 37 | SEQ ID NO: 52 | SEQ ID NO: 58 | SEQ ID NO: 54 |
| | TTGGACGGCTGGT | TCTCCAGATGAGGAA | TCCCAAGTCCAAA |
| | CCTCTAT | ACTAAGGACCTCATT | AGAAGGGATG |
| | | CATCG | |
| 38 | SEQ ID NO: 52 | SEQ ID NO: 59 | SEQ ID NO: 54 |
| | TTGGACGGCTGGT | AGGTCACTCAGACAG | TCCCAAGTCCAAA |
| | CCTCTAT | TCCCTCACATGTTC | AGAAGGGATG |
| 39 | SEQ ID NO: 52 | SEQ ID NO: 60 | SEQ ID NO: 54 |
| | TTGGACGGCTGGT | CTCAGACAGTCCCTC | TCCCAAGTCCAAA |
| | CCTCTAT | ACATGTTCCCCAG | AGAAGGGATG |
| 40 | SEQ ID NO: 52 | SEQ ID NO: 61 | SEQ ID NO: 54 |
| | TTGGACGGCTGGT | ATGAAGAGGTGGTTC | TCCCAAGTCCAAA |
| | CCTCTAT | AACTTTCTCCA | AGAAGGGATG |

A PCR primer set for amplifying RNAse L, PAP, Pim-1, Pim-2, Hepsin, PSMA, BK Virus or EBV RNA or DNA comprises at least one of the following sets of primer sequences: (1) SEQ ID NOS: 1 and 3; (2) SEQ ID NOS: 4 and 3; (3) SEQ ID NOS: 5 and 3; (4) SEQ ID NOS: 7 and 3; (5) SEQ ID NOS: 9 and 11; (6) SEQ ID NOS: 19 and 20; (7) SEQ ID NOS: 28 and 30; (8) SEQ ID NOS: 31 and 30; (9) SEQ ID NOS: 34 and 36; (9) SEQ ID NOS: 39 and 30; (10) SEQ ID NOS: 34 and 30; (11) SEQ ID NOS: 42 and 44; (12) SEQ ID NOS: 42 and 47; (13) SEQ ID NOS: 42 and 49; (14) SEQ ID NOS: 42 and 51; and (15) SEQ ID NOS: 52 and 54.

Any set of primers can be used simultaneously in a multiplex reaction with one or more other primer sets, so that multiple amplicons are amplified simultaneously.

A probe for binding to RNAse L, PAP, Pim-1, Pim-2, Hepsin, PSMA, BK Virus or EBV RNA or DNA comprises at least one of the following probe sequences: SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61.

Primer sets for simultaneously amplifying the RNAse L, PAP, Pim-1, Pim-2, Hepsin , PSMA, BK Virus or EBV DNA or DNA comprises a nucleotide sequence selected from the primer sets consisting of: Groups 1-40 of Table 2. Oligonucleotide probes for binding to the RNAse L, PAP, Pim-1, Pim-2, Hepsin, PSMA, BK Virus or EBV DNA or DNA DNA comprises a nucleotide sequence selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61.

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims. The contents of all references cited herein are incorporated by reference in their entireties.

What is claimed is:

1. An isolated nucleic acid sequence comprising a sequence selected from the group consisting of SEQ ID NOS: 1-61.

2. A method of hybridizing one or more isolated nucleic acid sequences comprising: a sequence selected from the group consisting of SEQ ID NOS: 1-61 to a RNAse L, PAP, Pim-1, Pim-2, Hepsin or PSMA sequence, further comprising contacting one or more isolated nucleic acid sequences to a sample comprising the RNAse L, PAP, Pim-1, Pim-2, Hepsin or PSMA sequence under conditions suitable for hybridization.

3. The method of claim 2, wherein the RNAse L, PAP, Pim-1, Pim-2, Hepsin or PSMA sequence is a genomic sequence, a template sequence or a sequence derived from an artificial construct.

4. The method of claim 2, further comprising isolating the hybridized RNAse L, PAP, Pim-1, Pim-2, Hepsin or PSMA sequence.

5. The method of claim 2, further comprising quantitating the hybridized RNAse L, PAP, Pim-1, Pim-2, Hepsin or PSMA sequence.

6. The method of claim 2, further comprising sequencing the hybridized RNAse L, PAP, Pim-1, Pim-2, Hepsin or PSMA sequence.

7. The method of claim 2, further comprising monitoring the presence of the hybridized RNAse L, PAP, Pim-1, Pim-2, Hepsin or PSMA sequence.

8. A primer set comprising at least one forward primer selected from the group consisting of SEQ ID NOS: 1, 4, 5, 7, 9, 19, 28, 31, 34, 39, 42 and 52 and at least one reverse primer selected from the group consisting of SEQ ID NOS: 3, 11, 20, 30, 36, 44, 47, 49, 51 and 54.

9. The primer set of claim 8, wherein the primer set is selected from the group consisting of: Groups 1-40 of Table 2.

10. A primer comprising a sequence selected from the group consisting of SEQ ID NOS: 1, 3, 4, 5, 7, 9, 11, 19, 20, 28, 30, 31, 34, 36, 39, 42, 44, 47, 49, 51, 52 and 54 for the production of cDNA from RNAse L, PAP, Pim-1, Pim-2, Hepsin or PSMA RNA.

11. A method of producing a nucleic acid product, comprising contacting one or more isolated nucleic acid sequences selected from the group consisting of SEQ ID NOS: 1, 3, 4, 5, 7, 9, 11, 19, 20, 28, 30, 31, 34, 36, 39, 42, 44, 47, 49, 51, 52 and 54 to a sample comprising a RNAse L, PAP, Pim-1, Pim-2, Hepsin or PSMA sequence under conditions suitable for nucleic acid polymerization.

12. The method of claim 11, wherein the nucleic acid product is an amplicon produced using at least one forward primer selected from the group consisting of SEQ ID NOS: 1, 4, 5, 7, 9, 19, 28, 31, 34, 39, 42 and 52, and at least one reverse primer selected from the group consisting of SEQ ID NOS: 3, 11, 20, 30, 36, 44, 47, 49, 51 and 54.

13. The method of claim 11, further comprising a probe that hybridizes to the nucleic acid product.

14. The probe of claim 13, wherein the probe comprises a sequence selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61.

15. The probe of claim 14, wherein the probe is labeled with a detectable label selected from the group consisting of: a fluorescent label, a chemiluminescent label, a quencher, a radioactive label, biotin and gold.

16. A method for detecting RNAse L, PAP, Pim-1, Pim-2, Hepsin or PSMA RNA or DNA in a sample, comprising:
a) contacting the sample with at least one forward primer comprising a sequence selected from the group consisting of SEQ ID NOS: 1, 4, 5, 7, 9, 19, 28, 31, 34, 39, 42 and 52, and at least one reverse primer comprising a sequence selected from the group consisting of SEQ ID NOS: 3, 11, 20, 30, 36, 44, 47, 49, 51 and 54 under conditions such that nucleic acid amplification occurs to yield an amplicon; and
b) contacting the amplicon with one or more probes comprising one or more sequences selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61, under conditions such that hybridization of the probe to the amplicon occurs;
wherein hybridization of the probe is indicative of the presence of RNAse L, PAP, Pim-1, Pim-2, Hepsin or PSMA in the sample.

17. The method of claim 16, wherein the RNAse L, PAP, Pim-1, Pim-2, Hepsin or PSMA DNA is a cDNA produced by contacting an RNAse L, PAP, Pim-1, Pim-2, Hepsin or PSMA RNA with a reverse transcriptase primer sequence selected from the group consisting of SEQ ID NOS: 1, 3, 4, 5, 7, 9, 11, 19, 20, 28, 30, 31, 34, 36, 39, 42, 44, 47, 49, 51, 52 and 54 under conditions suitable for the production of cDNA.

18. The method of claim 16, wherein each of the one or more probes is labeled with a different detectable label.

19. The method of claim 16, wherein the one or more probes are labeled with the same detectable label.

20. The method of claim 16, wherein the sample is selected from the group consisting of: blood, urine, serum, plasma, neoplastic or other tissue obtained from biopsies and samples specifically obtained from the prostate, cerebrospinal fluid, and the central nervous system.

21. The method of claim 16, wherein the sample is from a human.

22. The method of claim 16, wherein the sample is non-human in origin.

23. The method of claim 16, wherein the sample is derived from an inanimate object.

24. The method of claim 16, wherein the at least one forward primer, the at least one reverse primer and the one or more probes is selected from the group consisting of: Groups 1-40 of Table 2.

25. The method of claim 16, further comprising quantitating RNAse L, PAP, Pim-1, Pim-2, Hepsin or PSMA RNA or DNA in a sample.

26. A kit for detecting, quantitating and sequencing RNAse L, PAP, Pim-1, Pim-2, Hepsin or PSMA RNA or DNA in a sample, comprising one or more probes comprising a sequence selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61

27. The kit of claim 26, further comprising:
a) at least one forward primer comprising the sequence selected from the group consisting of SEQ ID NOS: 1, 4, 5, 7, 9, 19, 28, 31, 34, 39, 42 and 52; and
b) at least one reverse primer comprising the sequence selected from the group consisting of SEQ ID NOS: 3, 11, 20, 30, 36, 44, 47, 49, 51 and 54.

28. The kit of claim 27, further comprising a reverse transcriptase primer sequence selected from the group consisting of SEQ ID NOS: 1, 3, 4, 5, 7, 9, 11, 19, 20, 28, 30, 31, 34, 36, 39, 42, 44, 47, 49, 51, 52 and 54.

29. The kit of claim 26, further comprising reagents for quantitating, monitoring and/or sequencing RNAse L, PAP, Pim-1, Pim-2, Hepsin or PSMA RNA or DNA in the sample.

30. The kit of claim 26, wherein the one or more probes are labeled with different detectable labels.

31. The kit of claim 26, wherein the one or more probes are labeled with the same detectable label.

32. The kit of claim 26, wherein the at least one forward primer and the at least one reverse primer are selected from the group consisting of: Groups 1-40 of Table 2.

33. A kit for detecting and quantitating a targeted RNAse L, PAP, Pim-1, Pim-2, Hepsin or PSMA sequence derived from an artificial construct, such as a plasmid, comprising:
a) at least one forward primer comprising the sequence selected from the group consisting of SEQ ID NOS: 1, 4, 5, 7, 9, 19, 28, 31, 34, 39, 42 and 52;
b) at least one reverse primer comprising the sequence selected from the group consisting of SEQ ID NOS: 3, 11, 20, 30, 36, 44, 47, 49, 51 and 54; and
c) one or more probes comprising a sequence selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61.

34. A kit for binding, amplifying and sequencing RNAseL, PAP, PIM-1, PIM-2, Hepsin or PSMA RNA, DNA or proviral DNA in a sample, comprising:
a) at least one forward primer comprising the sequence selected from the group consisting of: SEQ ID NOS: 1, 4, 5, 7, 9, 19, 28, 31, 34, 39, 42 and 52;
b) at least one reverse primer comprising the sequence selected from the group consisting of: SEQ ID NOS: 3, 11, 20, 30, 36, 44, 47, 49, 51 and 54; and
c) reagents for the sequencing of amplified DNA fragments.

35. The kit of claim 34, further comprising reagents for quantitating and monitoring RNAseL, PAP, PIM-1, PIM-2, HEPSIN or PSMA RNA or DNA in a sample.

36. A method of diagnosing prostate cancer, comprising:
a) contacting a sample with at least one forward and reverse primer set selected from the group consisting of: Groups 1-40 of Table 2;
b) conducting an amplification reaction, thereby producing an amplicon; and
c) detecting the amplicon using one or more probes selected from the group consisting of: SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61; wherein the detection of an amplicon is indicative of the presence of RNAseL, PAP, PIM-1, PIM-2, HEPSIN or PSMA in the sample.

37. The method of claim 36, wherein the sample is selected from the group consisting of: blood, urine, serum, plasma, neoplastic or other tissue obtained from biopsies and samples specifically obtained from the prostate, cerebrospinal fluid, and the central nervous system.

38. A method for diagnosing prostate cancer, comprising:
a) contacting a sample with at least one forward and reverse primer pair selected from the group consisting of: Groups 1-40 of Table 2;
b) conducting a reverse transcriptase-polymerase chain reaction, a polymerase chain reaction or signal amplification;
c) detecting the generation of an amplified product using one or more probes selected from the group consisting of SEQ ID NOS: 2, 6, 8, 10, 12, 13, 14, 15, 16, 17, 18, 21, 22, 23, 24, 25, 26, 27, 29, 32, 33, 35, 37, 38, 40, 41, 43, 45, 46, 48, 50, 53, 55, 56, 57, 58, 59, 60 and 61; wherein the generation of an amplified product indicates the presence of RNAseL, PAP, PIM-1, PIM-2, HEPSIN or PSMA in the sample; and
d) determining expression levels of RNAseL, PAP, PIM-1, PIM-2, HEPSIN or PSMA RNA.
